(19) United States
(12) Patent Application Publication
(10) Pub. No.: US 2018/0243520 A1
(43) Pub. Date: Aug. 30, 2018

(54) Vaporizer Pod

(71) Applicant: Intrepid Brands, LLC, Louisville, KY (US)

(72) Inventors: David M. Johnson, Owensboro, KY (US); Charles H. Melander, Owensboro, KY (US); Barak Rigbi, Hayward, CA (US)

(21) Appl. No.: 15/904,790

(22) Filed: Feb. 26, 2018

Related U.S. Application Data
(60) Provisional application No. 62/466,647, filed on Mar. 3, 2017.
(60) Provisional application No. 62/463,986, filed on Feb. 27, 2017.

(51) Int. Cl. A61M 15/00; A61M 15/06

(52) U.S. Cl. CPC A61M 15/0063; A61M 15/06; A61M 15/0086; A61M 15/0001

(57) ABSTRACT

A vaporizer pod for use in a vaporizer is shown and described herein. The vaporizer pod defines a one-way channel therethrough, extending from a bottom end of the vaporizer pod to a top end. The vaporizer pod may include a valve disposed at the top end and a valve disposed at the bottom end, whereby the valves open the one-way channel as the user draws air through the vaporizer. The valves rest against and close a plurality of air channels to prevent the vaporizer liquid or wax from spilling out of the pod when the user is not drawing air through the vaporizer. The vaporizer pod may be reusable or disposable, whereby the reusable vaporizer pod includes a main body split into an upper and lower tube which the user may pull apart to load vaping material into the vaporizer pod. The disposable vaporizer pod includes a unitary main body.

### BACKGROUND

In some instances, it may be desirable to provide a vaporizer pod for use in a vaporizer, whereby the vaporizer pod supplies the vaping liquid to the vaporizer. Current vaporizer pods include permanently exposed holes in the top or the bottom of the pod with an absorbing material disposed inside the pod. Ideally, this absorbing material soaks up the vaporizing liquid and holds it inside the pod. Unfortunately, oftentimes the absorbing material does not hold the vaporizing liquid and it leaks out of the permanently exposed holes and either into the vaporizer itself or into the surrounding environment such as pant pockets or a carrying case. Therefore, a tremendous need exists to prevent a vaporizer pod from leaking vaporizing material when not in use.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a top perspective view of an exemplary reusable vaporizer pod;

FIG. 2 depicts a bottom perspective view of the reusable vaporizer pod of FIG. 1;

FIG. 3 depicts an exploded perspective view of the reusable vaporizer pod of FIG. 1;

FIG. 4 depicts another exploded perspective view of the reusable vaporizer pod of FIG. 1;

FIG. 5 depicts a perspective view of an upper tube and a screen separated from a lower tube of the reusable vaporizer pod of FIG. 1;

FIG. 6 depicts a perspective cross-sectional view taken along line 6-6 of FIG. 5;

FIG. 7 depicts an elevational cross-sectional view taken along line 7-7 of FIG. 1;

FIG. 8 depicts a top perspective view of an exemplary disposable vaporizer pod;

FIG. 9 depicts a bottom perspective view of the reusable vaporizer pod of FIG. 8;

FIG. 10 depicts an exploded perspective view of the reusable vaporizer pod of FIG. 8;

FIG. 11 depicts another exploded perspective view of the reusable vaporizer pod of FIG. 8;

FIG. 12 depicts a perspective cross-sectional view taken along line 12-12 of FIG. 8;

FIG. 13 depicts an elevational cross-sectional view taken along line 12-12 of FIG. 8;

FIG. 14A depicts a bottom perspective view of an exemplary valve for use in either the reusable vaporizer pod of FIG. 1 or the disposable vaporizer pod of FIG. 8;

FIG. 14B depicts is a side elevational view of the valve of FIG. 14A; and

FIG. 14C depicts is an elevational cross-sectional view taken along line 14C-14C of FIG. 14B.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Reusable Vaporizer Pod

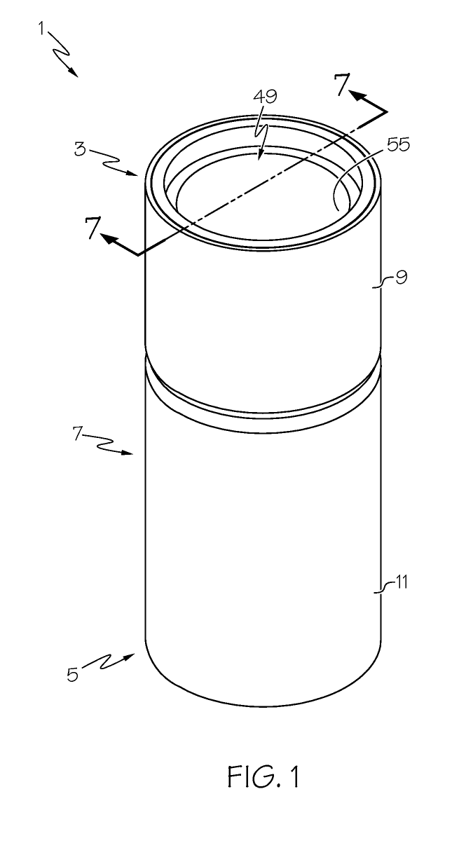

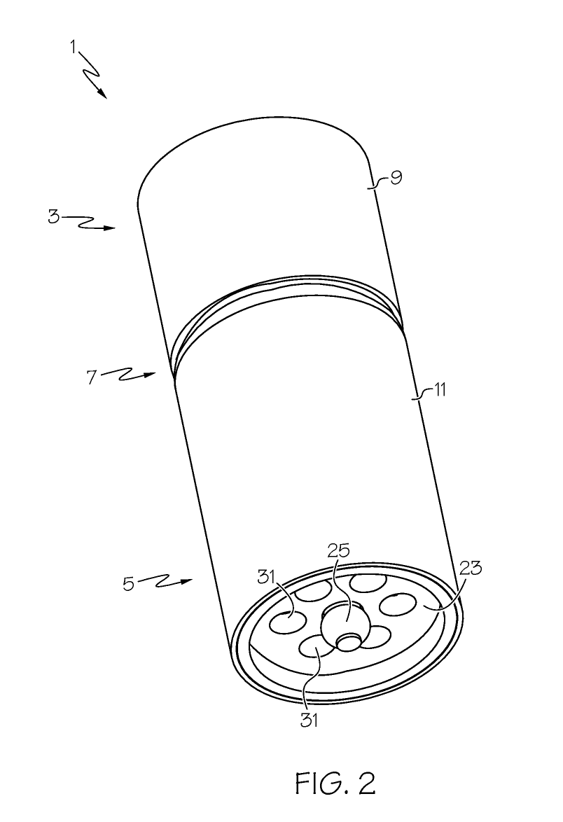

An exemplary reusable vaporizer pod (1) of the present invention is illustrated in FIGS. 1-7. Reusable vaporizer pod (1) is configured to receive either a liquid or a wax substance therein and be inserted into a vaporizer (not shown). Reusable vaporizer pod (1) extends from a top end (3) to a bottom end (5) and is generally cylindrical, whereby the air drawn through the vaporizer enters bottom end (5) of reusable vaporizer pod (1) and exits top end (3).

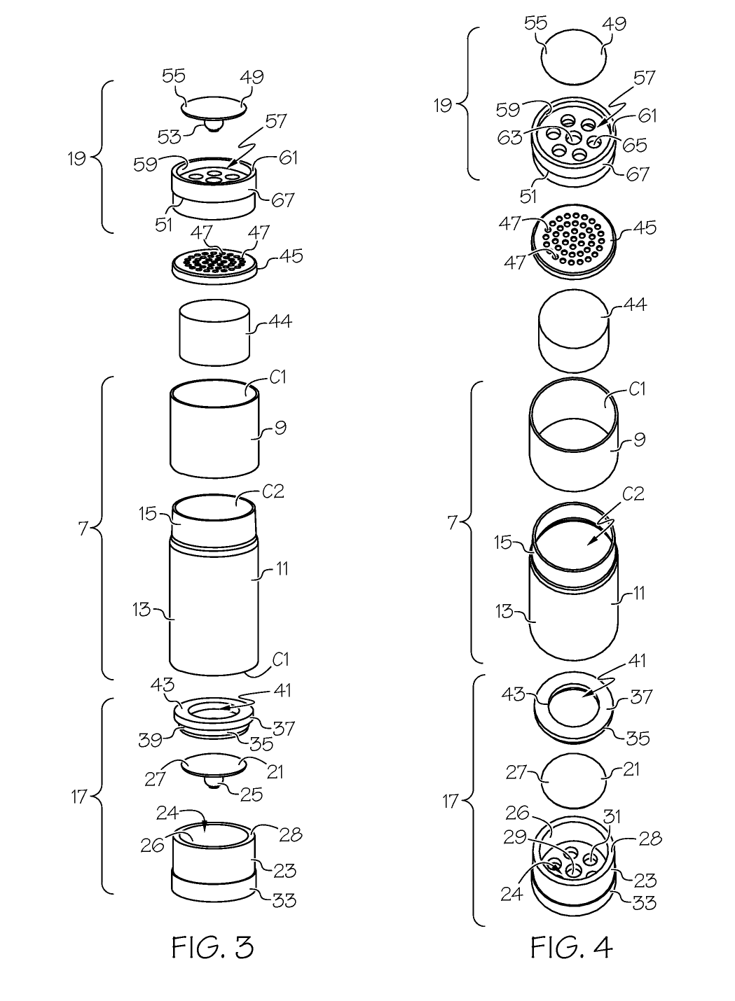

As shown in FIGS. 3 and 4, reusable vaporizer pod (1) includes a main body (7) comprised of an upper tube (9) and a lower tube (11). Upper tube (9) includes a generally cylindrical profile having a particular circumference (C1). Lower tube (11) includes a generally stepped cylindrical profile having a first area (13) with circumference (C1) and a second area (15) having a particular circumference (C2). Circumference (C2) is sized to allow circumference (C1) of upper tube (9) to slide down onto lower tube (11) and be removably secured thereto through a press fit frictional engagement.

Reusable vaporizer pod (1) further includes a lower valve assembly (17) disposed at bottom end (5) and an upper valve assembly (19) disposed at top end (3). In some versions of reusable vaporizer pod (1), lower valve assembly (17) is permanently secured to main body (7). In other versions, lower valve assembly (17) is removably secured to main body (7). Similarly, in some versions of reusable vaporizer pod (1), upper valve assembly (19) is permanently secured to main body (7). In other versions, upper valve assembly (19) is removably secured to main body (7).

Lower valve assembly (17) includes a lower valve (21) and a lower valve cap (23). In some versions of lower valve assembly (17), lower valve (21) is releasably coupled with lower valve cap (23). In other versions, lower valve (21) is permanently secured to lower valve cap (23). Some versions of lower valve (21) includes a bulb portion (25) extending from a flap portion (27). Lower valve cap (23) defines recess (24) by way of an inner surface (26). Inner surface (26) extends perpendicularly from an annular top surface (28). Inside recess (24), lower valve cap (23) includes a wall (30) which defines a central valve aperture (29) sized to receive bulb portion (25) therethrough. In some embodiments of removable vaporizer pod (1), bulb portion (25) and central valve aperture (29) are configured to allow the user to press on bulb portion (25) to release lower valve (21) from lower valve cap (23) and thus allow the user to clean the elements of removable vaporizer pod (1).

Lower valve cap (23) further defines a series of air channels (31) disposed proximate flap portion (27) when bulb portion (25) of lower valve (21) is coupled with lower valve cap (23). Flap portion (27) is biased toward air channels (31) when lower valve (21) is coupled with lower valve cap (23). However, flap portion (27) is configured such that the bias may be overcome when a user is drawing air through the vaporizer. Lower valve cap (23) further includes an annular rim (33) sized to engage first area (13) of lower tube (11) and fit therein through a press fit connection.

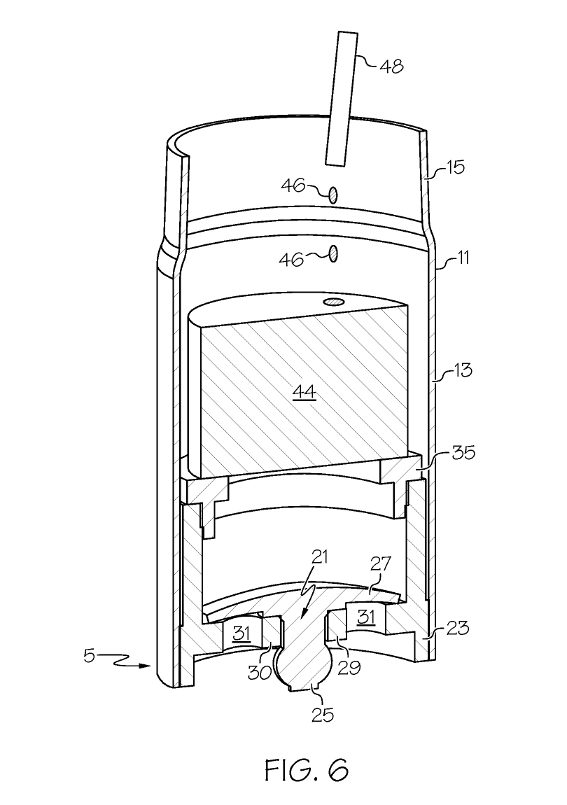

Lower valve assembly (17) further includes an inner cap (35), which includes a first section (37) having an annular rim (43) and a second section (39). Inner cap (35) further defines a channel (41) extending therethrough. As shown in FIG. 6, second section (39) is sized to fit into recess (24) of lower valve cap (23), whereby vapor drawn through reusable vaporizer pod (1) travels up through air channels (31), past lower valve (21), and through channel (41).

Once lower valve (21) is secured to lower valve cap (23) to form lower valve assembly (17), second section (39) of inner cap (35) is disposed into recess (24), thereby positioning first section (37) in an abutting relationship with annular top surface (28) of lower valve cap (23). Thereafter, lower valve assembly (17) and inner cap (35) are pressed into first area (13) of lower tube (11) to secure lower valve assembly (17) and inner cap (35) therein.

As shown in FIGS. 3 and 4, reusable vaporizer pod (1) further includes an absorbing material (44) and a screen (45). Absorbing material (44) may be formed from cellulose, glass frit, metal mesh, or any other type of material useful in absorbing or retaining liquid or wax substances to facilitate vaporizing of these materials. While absorbing material (44) is depicted in a cylindrical shape, absorbing material (44) may take the form of any shape and may be comprised of several different elements. As shown in FIG. 6, absorbing material (44) rests on annular rim (43) of inner cap (35). Screen (45) defines a plurality of holes (47) extending therethrough. As shown in FIG. 6, screen (45) is sized to fit inside upper tube (9) and abut lower tube (11).

Upper valve assembly (19) is generally similar to lower valve assembly (17) in that upper valve assembly (19) includes an upper valve (49) and an upper valve cap (51). Similar to lower valve (21), upper valve (49) includes a bulb portion (53) and a flap portion (55). Similar to lower valve cap (23), upper valve cap (51) includes an annular rim (67) and a recess (57) defined by an inner surface (59) extending from an annular top surface (61). Upper valve cap (51) further defines a central valve aperture (63) and a plurality of air channels (65) disposed within recess (57). Bulb portion (53) of upper valve (49) is sized to be received by central valve aperture (63) and thereby position flap portion (55) proximate air channels (65). In some embodiments of removable vaporizer pod (1), bulb portion (53) and central valve aperture (63) are configured to allow the user to press on bulb portion (53) to release upper valve (49) from upper valve cap (51) and thus allow the user to clean the elements of removable vaporizer pod (1).

Flap portion (55) is biased toward air channels (65) when upper valve is coupled with upper valve cap (51). However, flap portion (55) is configured such that the bias may be overcome when a user is drawing air through the vaporizer. As air is drawn through reusable vaporizer pod (1), flap portion (55) is lifted to expose air channels (65) and allow the air to pass therethrough. When air is not flowing through reusable vaporizer pod (1), flap portion (55) retracts to abut and overlay air channels (65), thus preventing any liquid or wax material from escaping through air channels (65).

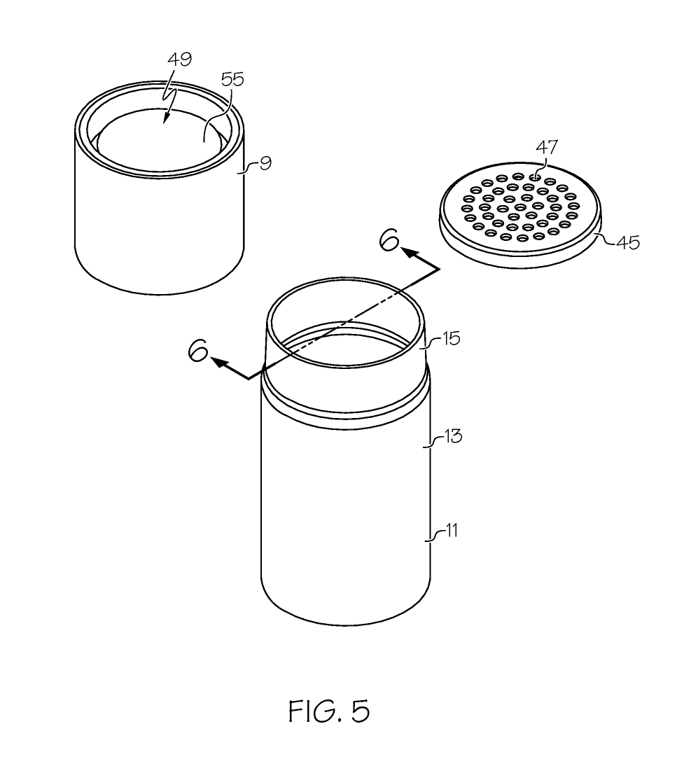

As shown in FIGS. 5 and 6, when upper valve assembly (19) is pressed into upper tube (9), annular rim (67) firmly holds upper valve assembly (19) within upper tube (9) via a press fit connection. Thereafter, a user is free to remove upper tube (9) along with upper valve assembly (19) to expose screen (45) and the remaining elements of reusable vaporizer pod (1). A user may then remove screen (45) to deposit vaporizing liquid or wax within lower tube (11), as shown with a vaporizing liquid (46) and dropper (48) in FIG. 6. Or alternatively, the user may deposit vaporizing liquid directly through screen (45) and allow the liquid to move through holes (47) and into lower tube (11). To reassemble reusable vaporizer pod (1), the user simply places screen (45) back onto the top of lower tube (11) and presses upper tube (9) onto lower tube (11), whereby upper tube (9) presses onto second area (15) and is held thereby via a frictional press fit engagement between second area (15) and upper tube (9).

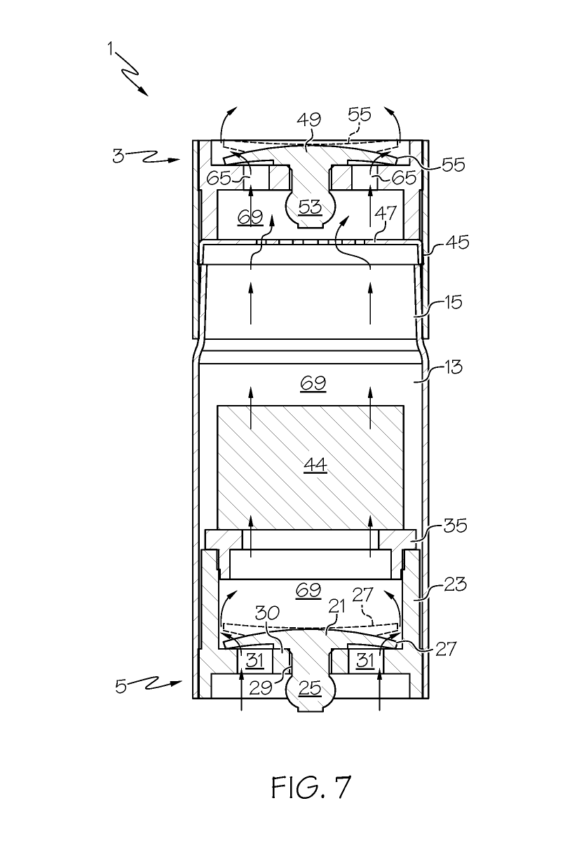

As shown in FIG. 7, reusable vaporizer pod (1) defines a unidirectional one-way channel (69) extending generally from bottom end (5) to top end (3). One-way channel (69) extends from air channels (31) of lower valve cap (23) through inner cap (35), lower tube (11), and upper tube (9), and out through air channels (65) of upper valve cap (51). One-way channel (69) transitions between a closed configuration and an open configuration, depending on the suction and air flow created by a user drawing air through the underlying vaporizer. When a user is drawing air through the vaporizer, one-way channel (69) is in the open configuration, at all other times, one-way channel (69) is in the closed position. In the closed configuration, flap portion (27) of lower valve (21) abuts air channels (31) and prevents any air from flowing in either direction through air channels (31). Similarly, flap portion (55) of upper valve (49) abuts air channels (65) in the closed position and prevents any air from flowing in either direction through air channels (65). In the open configuration, the suction created by the user and the flow of air through the underlying vaporizer acts to overcome the bias of each flap portion (27, 55), lifting flap portions (27,55) off air channels (31, 65) to open one-way channel (69).

One-way channel (69) affirmatively opens and closes in response to a user drawing air through the vaporizer. Inasmuch as one-way channel (69) is in the closed position by default, when the user is not using the vaporizer any vaping liquid or wax substance deposited within lower tube (11) is prevented from spilling or otherwise exiting reusable vaporizer pod (1). When reusable vaporizer pod (1) is not in use, one-way channel (69) remains closed in any orientation of reusable vaporizer pod (1) and thus can be laid horizontally on a surface or carried in a pocket without spilling the vaping materials.

II. Exemplary Disposable Vaporizer Pod

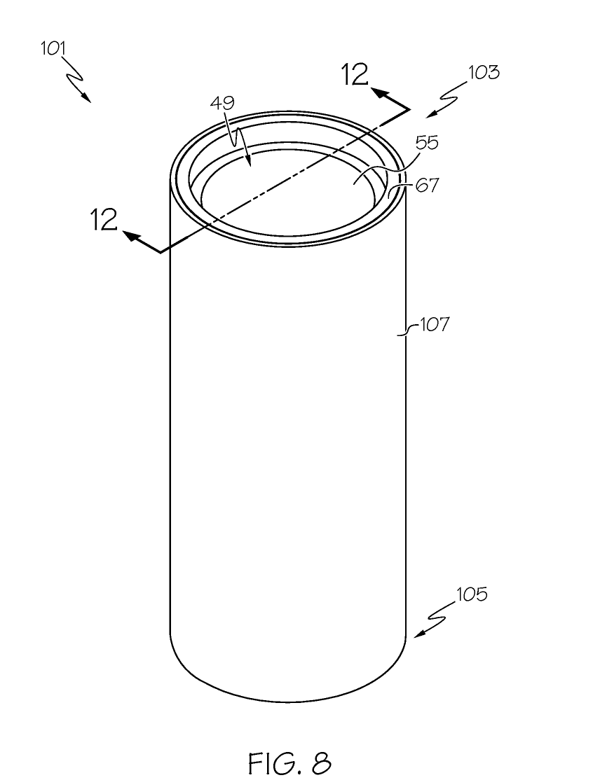

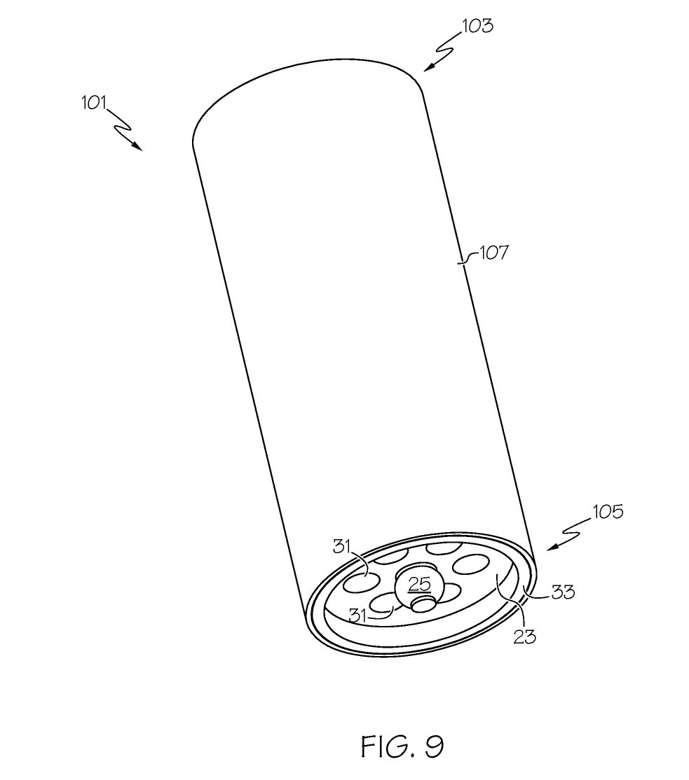

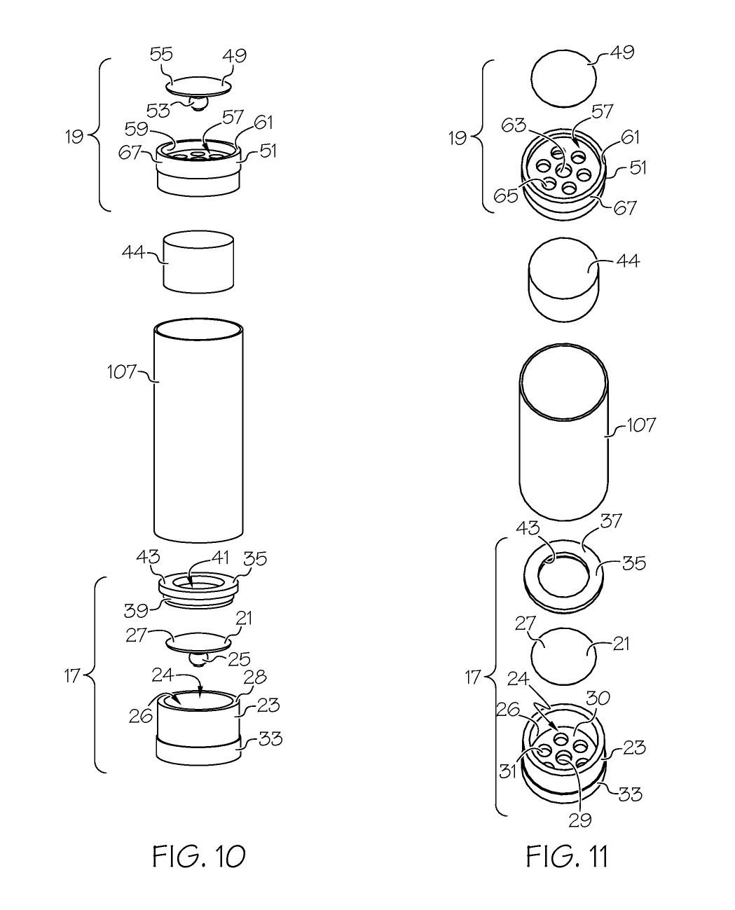

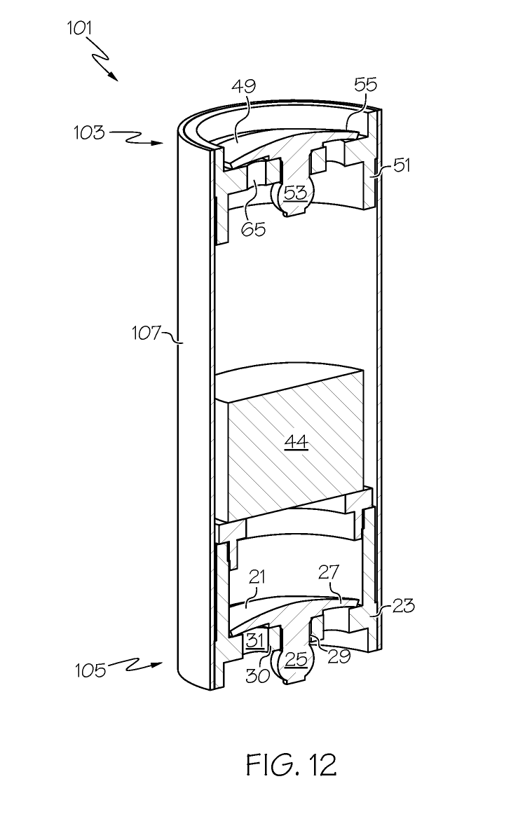

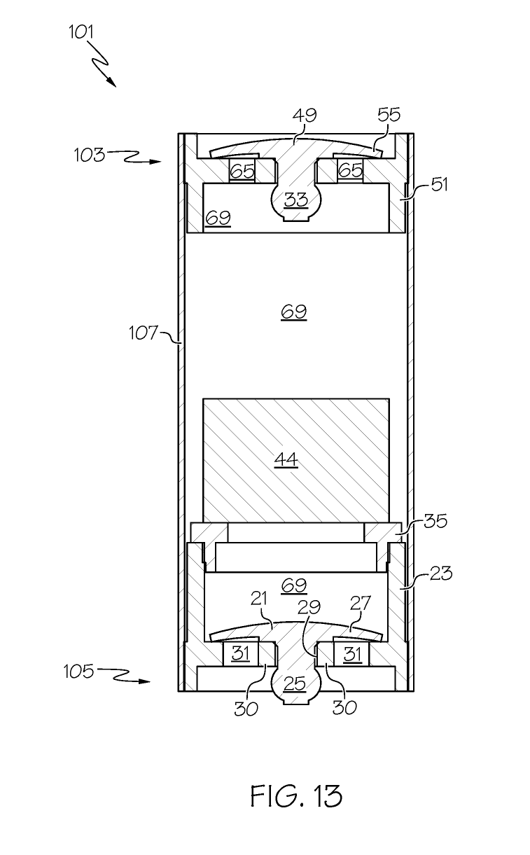

A disposable vaporizer pod (101) is shown in FIGS. 8-13 and is similar to reusable vaporizer pod (1) in many respects, extending from a top end (103) to a bottom end (105) and being generally cylindrical. However, rather than main body (7) comprising upper tube (9) and lower tube (11), disposable vaporizer pod (101) includes a main body (107) with a unitary design and free of any separation between upper and lower areas. The unitary structure of main body (107) generally prevents the user from accessing the inner areas of disposable vaporizer pod (101) and refilling the inner areas with vaporizer liquid or wax. At top end (103), annular rim (67) of upper valve cap (51) firmly abuts the inner surface of main body (107) to secure upper valve assembly (19) to main body (107) and block access to the internal area of disposable vaporizer pod (101) and absorbing material (44). At bottom end (105), annular rim (33) of lower valve cap (23) firmly abuts the inner surface of main body (107) to secure lower valve assembly (17) to main body (107) and block access to the internal area of disposable vaporizer pod (101).

Prior to securing either upper valve assembly (19) or lower valve assembly (17) to main body (107), a manufacturer or other party loads the inner area of main body (107) with either a vaping liquid or wax. Thereafter, main body (107) is sealed by pressing either upper valve assembly (19) or lower valve assembly (17) into position. Disposable vaporizer pod (101) may be packaged, shipped, or otherwise move through commerce without spilling the vaping material as one-way channel (69) acts to hold the liquid/wax in disposable vaporizer pod (101). When disposable vaporizer pod (101) ultimately reaches the end user, the user loads disposable vaporizer pod (101) into an appropriate vaporizer and draws on the vaporizer to open up one-way channel (69) and ingest the vaporized liquid or wax. Once the material within disposable vaporizer pod (101) is exhausted, the user removes disposable vaporizer pod (101) from the vaporizer and discards it.

III. Exemplary Vaporizer Pod Valve

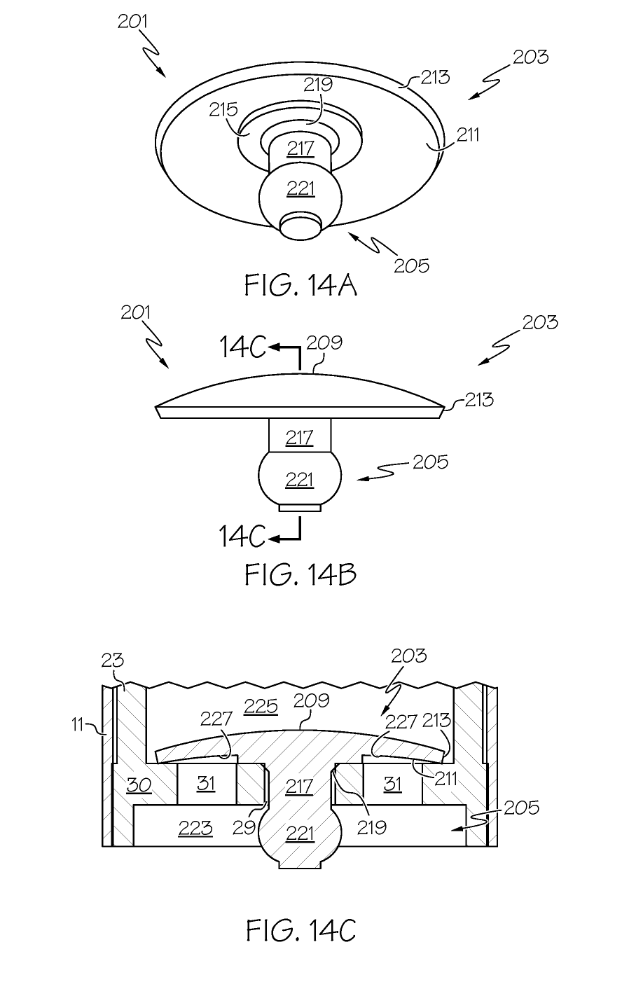

FIGS. 14A-14C depict a valve (201) for use in reusable vaporizer pod (1) and/or disposable vaporizer pod (101). Valve (201) may be comprised of a resilient, flexible material such as polymers or engineered plastic, or may be comprised of rigid material such as metal or glass. In those version of valve (201) where a rigid material is used, a flexible sleeve or other element may be used in central valve aperture (29, 63) to allow valve (201) to couple with main body (7). The diameter of valve (201) is based on the size of main body (7), as it must fit and seal the internal chamber of reusable vaporizer pod (1) and/or disposable vaporizer pod (101).

Valve (201) is similar to the previously described lower valve (21) and upper valve (49). Valve (201) includes a generally mushroom-shaped profile with a flap portion (203) extending from a bulb portion (205). Flap portion (203) includes a convex top surface (207) peaking at an apex (209), a concave bottom surface (211), and a side surface (213) extending therebetween and around the periphery of valve (201). A ring (215) extends from bottom surface (211) downwardly toward a proximal end of a shaft (217) of bulb portion (205). A chamfered surface (219) extends between ring (215) and shaft (217). A bulb (221) is disposed at a distal end of shaft (217), whereby bulb (221) includes a larger cross-sectional diameter than the cross-sectional diameter of shaft (217).

As shown in FIG. 14C, when valve (201) is engaged with lower valve cap (23) of reusable vaporizer pod (1), bulb (221) is disposed on a first side (223) of wall (30) while flap portion (203) is disposed on a second side (225). Chamfered surface (219) is sized and positioned to abut the corner of the portion of wall (30) defining and surrounding central valve aperture (29) and press against this corner to seal central valve aperture (29) to prevent vapor from passing therethrough. Ring (215) is oriented to press against wall (30) in furtherance of preventing vapor from passing through central valve aperture (29).

Inasmuch as the vapor is blocked from traversing central valve aperture (29) by the ring (215) and chamfered surface (219), conversely, the curved nature of flap portion (203) defines a pocket (227) above each air channel (31) to prevent flap portion (203) from being pulled or sucked into each air channel (31). The curved nature of flap portion (203) also provides additional area for the vapor to build before the user inhales and raises flap portion (203) through the user's suction force. The curve of flap portion (203) directs bottom surface (211) and side surface (213) out over each air channel (31) and onto the surface of wall (30), sealing each air channel (31). As the user draws vapor through the vaporizer, the suction forces lift flap portion (203) to open fluid communication between first side (223) of wall (30) and second side (225) of wall (30), allowing the vapor to flow therethrough.

While valve (201) is shown and described in connection with lower valve cap (23) of reusable vaporizer pod (1), valve (201) may be used in a similar manner with lower valve cap (23) of disposable vaporizer pod (101), upper valve cap (51) of reusable vaporizer pod (1), or upper valve cap (51) of disposable vaporizer pod (101).

In order to clean or change valve (201), the user presses on bulb (221) to retract bulb (221) up through wall (31) from first side (223) to second side (225) to thereby release valve (201) from lower valve cap (23) or upper valve cap (51) as desired.

IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A vaporizer pod comprising configured to be received in a vaporizer, the vaporizer pod comprising: (a) a main body extending from a top end to a bottom end; and (b) a lower valve assembly, wherein the lower valve assembly is secured to the main body proximate the bottom end, wherein the lower valve assembly comprises a lower valve, wherein the lower valve is resilient.

### EXAMPLE 2

The vaporizer pod of Example 1 or any of the following Examples, wherein the lower valve assembly comprises a lower valve cap, wherein the lower valve is removably coupled to the lower valve cap.

### EXAMPLE 3

The vaporizer pod of any of the previous or any of the following Examples, wherein the lower valve cap having a first side and a second side, wherein the lower valve cap defines a plurality of air channels therethrough and extending between the first side and the second side.

### EXAMPLE 4

The vaporizer pod of any of the previous or any of the following Examples, wherein the lower valve comprises a flap portion, wherein the flap portion is biased toward the plurality of air channels when the lower valve is coupled with the lower valve cap.

### EXAMPLE 5

The vaporizer pod of any of the previous or any of the following Examples, wherein the lower valve comprises a bulb and a shaft, wherein the shaft extends between the flap portion and the bulb.

### EXAMPLE 6

The vaporizer pod of any of the previous or any of the following Examples, wherein the bulb is disposed on the first side of the lower valve cap when the lower valve is coupled with the lower valve cap, wherein the flap portion is disposed on the second side of the lower valve cap when the lower valve is coupled with the lower valve cap.

### EXAMPLE 7

The vaporizer pod of any of the previous or any of the following Examples, further comprising an inner cap disposed in the main body, wherein the inner cap defines a channel, wherein the inner cap abuts the lower valve cap in the main body.

### EXAMPLE 8

The vaporizer pod of any of the previous or any of the following Examples, further comprising an absorbing material disposed in the main body, wherein the absorbing material abuts the lower valve cap in the main body.

### EXAMPLE 9

The vaporizer pod of any of the previous or any of the following Examples, wherein the main body comprises an upper tube and a lower tube, wherein the upper tube is removably coupled with the lower tube, wherein the lower valve assembly is secured to the lower tube.

### EXAMPLE 10

The vaporizer pod of any of the previous or any of the following Examples, further comprising an upper valve assembly, wherein the upper valve assembly comprises an upper valve removably coupled to an upper valve cap.

### EXAMPLE 11

The vaporizer pod of any of the previous or any of the following Examples, wherein the upper valve assembly is secured to the upper tube.

### EXAMPLE 12

The vaporizer pod of any of the previous or any of the following Examples, further comprising a screen, wherein the screen is disposed between the upper valve assembly and the lower valve assembly.

### EXAMPLE 13

A vaporizer pod comprising: (a) a main body extending from a top end to a bottom end; (b) an upper valve disposed proximate the top end; and (c) a lower valve disposed proximate the bottom end.

### EXAMPLE 14

The vaporizer pod of any of the previous or any of the following Examples, further comprising an upper valve cap, wherein the upper valve is removably secured to the upper valve cap.

### EXAMPLE 15

The vaporizer pod of any of the previous or any of the following Examples, further comprising a lower valve cap, wherein the lower valve is removably secured to the lower valve cap.

### EXAMPLE 16

The vaporizer pod of any of the previous or any of the following Examples, further comprising an absorbable material disposed in the main body between the upper valve and the lower valve.

### EXAMPLE 17

The vaporizer pod of any of the previous or any of the following Examples, further comprising an inner cap disposed in the main body and abutting the lower valve cap and the absorbable material.

### EXAMPLE 18

The vaporizer pod of any of the previous or any of the following Examples, wherein the upper valve cap defines a central valve aperture, wherein a portion of the upper valve is disposed within the central valve aperture.

### EXAMPLE 19

The vaporizer pod of any of the previous or any of the following Examples, wherein the upper valve cap defines a plurality of air channels disposed about the central valve aperture.

### EXAMPLE 20

The vaporizer pod of any of the previous or any of the following Examples, wherein the upper valve includes a flap portion, wherein the flap portion includes a convex surface having an apex.

VI. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

We claim:

1. A vaporizer pod configured to be received in a vaporizer, the vaporizer pod comprising:
(a) a main body extending from a top end to a bottom end; and
(b) a lower valve assembly, wherein the lower valve assembly is secured to the main body proximate the bottom end, wherein the lower valve assembly comprises a lower valve, wherein the lower valve is resilient.

2. The vaporizer pod of claim 1, wherein the lower valve assembly comprises a lower valve cap, wherein the lower valve is removably coupled to the lower valve cap.

3. The vaporizer pod of claim 2, wherein the lower valve cap having a first side and a second side, wherein the lower valve cap defines a plurality of air channels therethrough and extending between the first side and the second side.

4. The vaporizer pod of claim 3, wherein the lower valve comprises a flap portion, wherein the flap portion is biased toward the plurality of air channels when the lower valve is coupled with the lower valve cap.

5. The vaporizer pod of claim 4, wherein the lower valve comprises a bulb and a shaft, wherein the shaft extends between the flap portion and the bulb.

6. The vaporizer pod of claim 5, wherein the bulb is disposed on the first side of the lower valve cap when the lower valve is coupled with the lower valve cap, wherein the flap portion is disposed on the second side of the lower valve cap when the lower valve is coupled with the lower valve cap.

7. The vaporizer pod of claim 6, further comprising an inner cap disposed in the main body, wherein the inner cap defines a channel, wherein the inner cap abuts the lower valve cap in the main body.

8. The vaporizer pod of claim 7, further comprising an absorbing material disposed in the main body, wherein the absorbing material abuts the lower valve cap in the main body.

9. The vaporizer pod of claim 8, wherein the main body comprises an upper tube and a lower tube, wherein the upper tube is removably coupled with the lower tube, wherein the lower valve assembly is secured to the lower tube.

10. The vaporizer pod of claim 9, further comprising an upper valve assembly, wherein the upper valve assembly comprises an upper valve removably coupled to an upper valve cap.

11. The vaporizer pod of claim 10, wherein the upper valve assembly is secured to the upper tube.

12. The vaporizer pod of claim 11, further comprising a screen, wherein the screen is disposed between the upper valve assembly and the lower valve assembly.

13. A vaporizer pod comprising:
(a) a main body extending from a top end to a bottom end;
(b) an upper valve disposed proximate the top end; and
(c) a lower valve disposed proximate the bottom end.

14. The vaporizer pod of claim 13, further comprising an upper valve cap, wherein the upper valve is removably secured to the upper valve cap.

15. The vaporizer pod of claim 14, further comprising a lower valve cap, wherein the lower valve is removably secured to the lower valve cap.

16. The vaporizer pod of claim 15, further comprising an absorbable material disposed in the main body between the upper valve and the lower valve.

17. The vaporizer pod of claim 16, further comprising an inner cap disposed in the main body and abutting the lower valve cap and the absorbable material.

18. The vaporizer pod of claim 14, wherein the upper valve cap defines a central valve aperture, wherein a portion of the upper valve is disposed within the central valve aperture.

19. The vaporizer pod of claim 18, wherein the upper valve cap defines a plurality of air channels disposed about the central valve aperture.

20. The vaporizer pod of claim 19, wherein the upper valve includes a flap portion, wherein the flap portion includes a convex surface having an apex.
